(19) United States
(12) Patent Application Publication
(10) Pub. No.: US 2007/0282385 A1
(43) Pub. Date: Dec. 6, 2007

(54) LOCKOUT CONNECTOR ARRANGEMENT FOR IMPLANTABLE MEDICAL DEVICE

(75) Inventors: Martin A. Rossing, Coon Rapids, MN (US); Stephen L. Bolea, Watertown, MN (US); Aaron Hjelle, Champlin, MN (US); James T. Henry, Blaine, MN (US); Brad D. Pedersen, Edina, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Maple Grove, MN (US)

(21) Appl. No.: 11/836,042

(22) Filed: Aug. 8, 2007

Related U.S. Application Data
(63) Continuation of application No. 11/168,238, filed on Jun. 27, 2005.
(60) Provisional application No. 60/584,647, filed on Jun. 30, 2004.

(51) Int. Cl. A61N 1/02

(52) U.S. Cl. 607/37

(57) ABSTRACT

A lockout connector arrangement for implantable medical devices having at least one port for receiving a non-cardiac lead connector selectively permits only certain electrical leads to be connected to the implantable medical device. A lead connector pin of a non-cardiac lead connector is specially designed to be larger than a DF-1 lead connector pin, but smaller than an IS-1 lead connector pin. A corresponding header of implantable pulse generator has a connector port for a non-cardiac lead with a proximal-most portion that is larger than the DF-1 lead connector pin, but smaller than the IS-1 lead connector pin; and otherwise generally consistent with the other dimensions of an ISO standard IS-1 pacemaker lead connector.

### CROSS-REFERENCES TO RELATED APPLICATIONS

The present application is a continuation of U.S. patent application Ser. No. 11/168,238 (Attorney Docket No.: 001410US), filed Jun. 27, 2005, which claims the benefit of U.S. Patent Application Ser. No. 60/584,647 (Attorney Docket No. 021433-001400US), filed Jun. 30, 2005, the full disclosures of which are incorporated herein by reference.

### BACKGROUND OF THE INVENTION

1. Field of the Invention

This invention relates to implantable electrical medical devices used to stimulate the heart, other tissue, and nerves, to control the functioning of the particular organ or bodily function. More particularly, the present invention is directed to electrical lead connector arrangements for implantable medical devices that selectively permit only certain electrical leads to be connected to the implantable medical device.

2. Background of the Invention

Implantable pulse generator medical devices are well known in the art, and include medical devices such as pacemakers, defibrillators, baroreflex activation devices and muscle and nerve stimulators. Generally, these medical electrical devices comprise an implantable pulse generator unit and an electrical lead or leads connected to one or more electrodes. The electrode may be placed adjacent to a particular part of the human body, such as within the myocardial tissue of the heart, within a vein or proximate any other tissue to be stimulated and/or sensed. The electrode, which is attached at the distal end of the lead, is attached to the appropriate location in the human body, and the proximal end of the lead is connected to a lead connector assembly of the implantable pulse generator. The lead connector assembly, sometimes referred to as a header, enables the lead to be mechanically and electrically connected to circuitry within the implantable pulse generator.

The header of an implantable pulse generator typically has a plurality of connector ports to which a plurality of leads may be connected. For pacemakers and defibrillators, these connector ports are either high voltage ports for receiving high voltage electrical lead connectors of a defibrillation electrode or low voltage connector ports for receiving electrical lead connectors of a sensing/pacing electrode. For other types of tissue stimulation devices, the connector ports are typically low or moderate voltage connector ports for receiving electrical lead connectors to connect to tissue sensing and/or stimulation electrodes.

For implantable pulse generators having a plurality of ports and a plurality of leads, it is possible for a particular lead to be inserted into an improper port. If this were to happen, the delivery of stimulation pulses through an improperly connected lead would not provide the intended therapy and could be potentially damaging or fatal to a patient. A non-cardiac stimulation lead connected to a pacing port would likely deliver an ineffective therapy, and could even have the dramatic consequence of inducing fibrillation in the patient; however, the non-cardiac stimulation lead most likely would not be damaged due to the relatively low voltage of the pacing stimulation pulses. A potentially more dangerous situation would arise if a low or moderate voltage lead were to be connected to a connector port for a high voltage defibrillation electrode. Not only would the unintended delivery of a high voltage defibrillation shock of up to 750 V through a pacing or stimulation electrode designed for voltages of less than 5 V likely cause damage to that low or moderate voltage electrical lead, the consequences for the unintended delivery of such a shock could be damaging or even fatal to a patient, even if fibrillation were not induced as a result of the shock.

To prevent defibrillator leads and pacer leads from being connected to the improper port, the International Standards Organization (ISO) developed standards for the pacer lead connector and the pacer port or cavity, as well as standards for the defibrillator lead connector and defibrillator port or cavity. The standard for the defibrillator connector and cavity is ISO 11318 and the standard for the pacemaker connector and cavity is ISO 5841-3, both of which are incorporated herein by reference. The standard pacer port is referred to as an IS-1 port and the standard defibrillator port is referred to as a DF-1 port. If the ISO standards are followed for these structures, then a lead made in accordance with one of the standards cannot be connected to a port constructed in accordance with the other of the standards. Hence, a pacer lead made according to the ISO standard (5841-3) will not be able to be connected to a defibrillator lead connector that was made according to the ISO standard (11318). The details of these ISO standards are hereby incorporated by reference.

Although the ISO standards provide guidance for defibrillators and pacers to ensure that the lead connectors cannot be operably connected to the improper port in these two types of implantable medical devices, the problem of how to avoid similar improper connections for other types of tissue stimulation leads is not addressed.

U.S. Pat. No. 6,044,302 describes a multiport header arrangement for a cardiac rhythm management device includes at least one standard port and a separate port for a left ventricular access lead. The left ventricular access lead can only be electrically and mechanically coupled to the proper port. Standard IS-1 and DF-1 leads cannot be electrically or mechanically coupled to the port for the left ventricular access lead. The lockout solution described in this patent requires the left ventricular access lead to have a smaller diameter than either the IS-1 or DF-1 leads so that the larger leads will not fit in the smaller connector port. The patent requires the physician to realize that the smaller left ventricular access lead has been improperly inserted into a larger IS-1 or DF-1 port because of the difficulty in locking down the smaller diameter lead connector with a set screw that secure the lead connector into the port.

U.S. Pat. No. 6,705,900 describes an improved connection system for coupling a device such as a pacemaker, cardioverter, defibrillator, nerve stimulator, muscle stimulator, implantable monitor or other medical device to a medical lead that features a coupling member, which includes an inner lumen sized to form a press fit around the proximal end of the lead body and has connector means to enable a connector pin at the proximal end of the lead to mechanically and electrically couple to a device. While this system provides a solution for adapting one type of lead to be used in a different type of connector port, it does not provide a solution to the problem of improperly inserting one type of lead in a different type of connector port.

Although existing standards have worked well for addressing the problems of proper connection of leads to implantable pulse generators for cardiac stimulation devices, there is a need for a more general solution for addressing the problems of proper connection of leads to implantable pulse generators for other types of tissue stimulation devices.

### BRIEF SUMMARY OF THE INVENTION

The present invention is a lockout connector arrangement for implantable medical devices having at least one port for receiving a non-cardiac lead connector that selectively permits only certain electrical leads to be connected to the implantable medical device. Specifically, a lead connector pin of a non-cardiac lead connector is specially designed to be larger than the lead connector pin of a DF-1 defibrillation lead connector port, but smaller than the lead connector pin of an IS-1 pacemaker lead connector port. A corresponding header is provided for an implantable pulse generator in which a connector port for a non-cardiac lead has a proximal-most portion that is larger than the lead connector pin of a DF-1 defibrillation lead, but smaller than the lead connector pin of an IS-1 pacemaker lead. While providing for effective lockout operation, the overall dimensions of the remainder of the lead connector of a preferred embodiment of the present invention remain generally consistent with the IS-1 standards to permit the non-cardiac connector port and connectors to be manufactured with minimal changes to existing header and lead designs.

As a result of the design of the connector arrangement of the present invention, the non-cardiac lead cannot be mechanically or electrically connected to a DF-1 defibrillation port, thus effectively barring the potential harm that could be done if a high-energy defibrillation pulse were delivered to a non-cardiac lead. Conversely, a DF-1 defibrillation lead cannot be inadvertently electrically connected to a non-cardiac port on the implantable pulse generator. While an IS-1 pacemaker lead could be mechanically inserted into the non-cardiac lead port of a header for an implantable pulse generator in accordance with the present invention, the electrical contact arrangement within the non-cardiac lead port prevents any inadvertent electrical connection from being effectively made. Conversely, the non-cardiac stimulation lead pin cannot make effective electrical connection with an IS-1 pacemaker lead port. Thus, the potential harm caused by a tissue stimulation therapy pulse being delivered to cardiac tissue through a pacing lead that uses an IS-1 standard, or a pacing stimulation therapy pulse being delivered to a non-cardiac lead, is effectively obviated.

### BRIEF DESCRIPTION OF THE DRAWINGS

FIG. 1 is a perspective view of a medical electrical implantable pulse generator and associated electrical leads.

FIG. 2 is a cross-sectional view of the IS-1 pacemaker lead connector that meets the ISO 5841-3 standard.

FIG. 3 is a cross-sectional view of the IS-1 pacemaker lead connector port that meets the ISO 5841 standard.

FIG. 4 is a cross-sectional view of the DF-1 defibrillator lead connector that meets the ISO 11318:2002 standard.

FIG. 5 is a cross-sectional view of the DF-1 defibrillator lead connector port that meets the ISO 11318:2002 standard.

FIG. 6 is a cross-sectional view of the non-cardiac lead connector in accordance with the present invention.

FIG. 7 is a cross-sectional view of the non-cardiac lead connector port in accordance with the present invention that interfaces with the non-cardiac lead connector as shown in FIG. 6.

### DETAILED DESCRIPTION OF THE INVENTION

An implantable pulse generator device typically includes an electrical medical device such as a pacemaker, cardioverter, defibrillator, baroreflex activation device, nerve stimulator, muscle stimulator, implantable monitor or other medical device and one or more electrical leads. Typically, the pulse generator device comprises a case and a header attached to the case. The case typically contains the electronics and the power source (usually a battery) for the implantable pulse generator. The leads are connected to the implantable pulse generator through ports in the header.

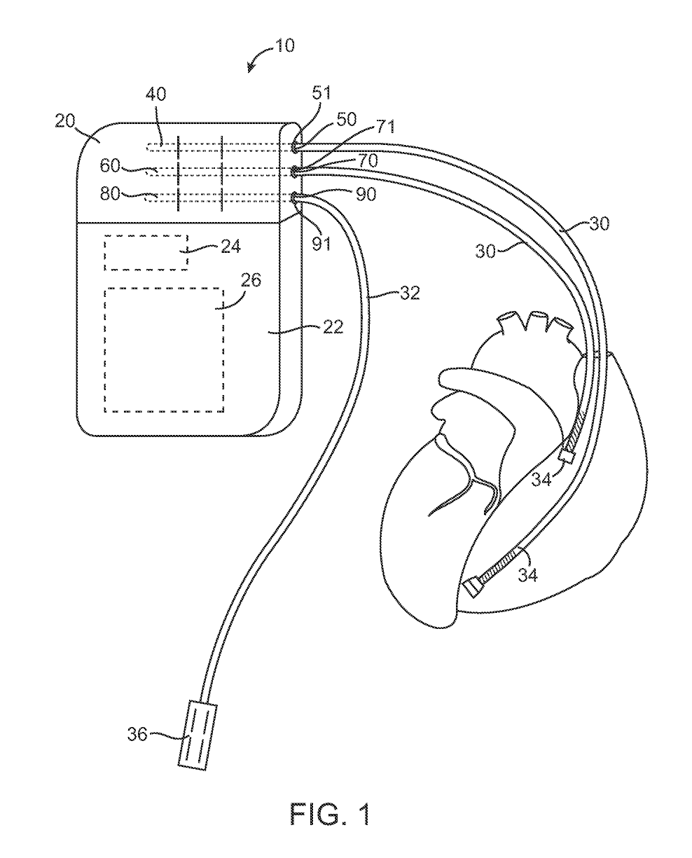

Referring to FIG. 1, there is shown an implantable pulse generator device 10 that is comprised of a header 20 and a case 22 containing a power source 24 and electronics 26. The header portion 20 of the implantable pulse generator device 10 is typically formed of a molded thermoplastic material, such as an acrylic material, and includes a plurality of ports 50 (pacing), 70 (defibrillation), and 90 (non-cardiac). While the number of ports shown in this embodiment is three, a greater or lesser number of ports is contemplated by the scope of the present invention. Each port 50, 70, 90 includes a corresponding orifice 51, 71, 91, which is the entry point to the port 50, 70, 90 and the interior of the header 20. Electrical leads 30, 32 connect the implantable pulse generator device 10 to electrodes 34, 36 typically located at their distal end that are positioned proximate a particular location in the body to be stimulated or sensed. The electrical leads 30, 32 are connected to the header 20 through the appropriate orifice 51, 71, 91 of the corresponding port 50, 70, 90 by way of a lead connector 40, 60 or 80. As will be described, a given lead connector 40, 60, 80 is designed to be inserted into a corresponding one of the ports 50, 70, 90 and mechanically and electrically couples the associated lead 30, 32 with the header 20.

The electrical leads may be cardiac leads 30 designed in accordance with either the IS-1 or DF-1 standard, or other types of cardiac leads 30, such as the left ventricular lead described in U.S. Pat. No. 6,044,302, or may be non-cardiac leads 32 that are intended for stimulation and/or sensing of tissue or organs other than the heart. In a preferred embodiment of the present invention, the non-cardiac lead connector and lead connector port are adapted for a non-cardiac lead 32 that includes a non-cardiac stimulation electrode 36. One such example of a non-cardiac stimulation electrode is a baroreflex activation lead and electrode for baroreflex activation, such as shown in U.S. Pat. No. 6,522,926 and U.S. Publ. Appl. Nos. 2003/0060857A1 and 2004/0010303A1, the disclosures of which are hereby incorporated by reference. Alternatively, the non-cardiac lead connector and lead connector port of the present invention may be utilized for any non-cardiac stimulation application, such as nerve, muscle or other tissue or organ stimulation.

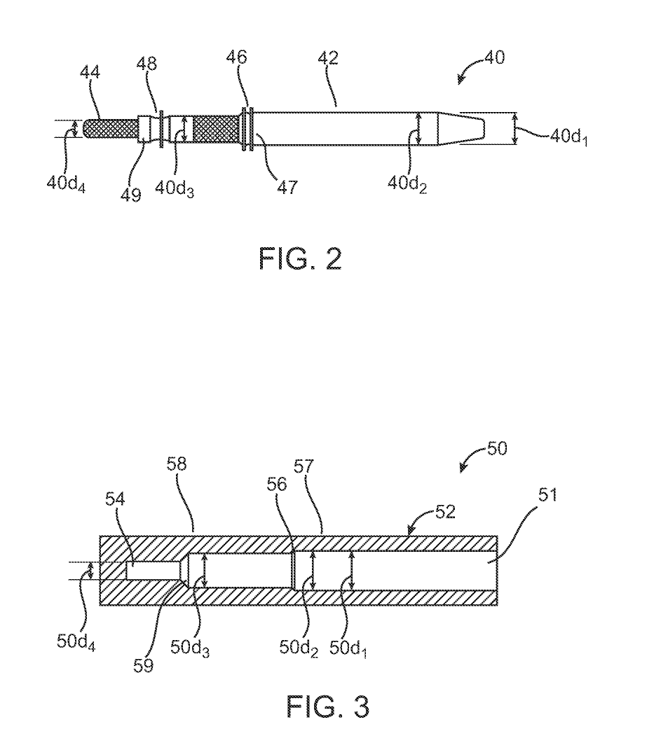

FIG. 2 is a cross-sectional view of the pacemaker lead connector 40 that meets the ISO 5841-3 standard. The lead connector 40 for the cardiac lead 30 comprises a lead connector body 42 and a lead connector pin 44. The lead connector pin 44 is located at the proximal end of the lead connector 40 and, when locked in place in the port 50, forms a mechanical and physical connection between the lead connector 40 and the header 20. The lead connector pin 44 is made of conductive material.

The lead connector body 42 has a number of different diameters, as the lead connector body 42 tapers towards the lead connector pin 44. The diameter 40d₁, of the lead connector body 42 proximate the lead is 3.1+/−0.3 millimeters. The diameter of the main section of the lead connector body 42, 40d₂, is 3.23+/−0.1 millimeters. This section of the lead connector body 42 extends up to the first shoulder 46, where at least one sealing ring 47 is located. At the first shoulder 46, the lead connector body 42 tapers to a diameter 40d₃ of 2.66+/−0.03 millimeters to 2.66+/−0.05 millimeters. A second sealing area with at least one sealing ring 48 precedes the second shoulder 49 of the lead connector body 42. At the second shoulder 49, the lead connector body 42 tapers again such that the lead connector pin 44 is formed with a diameter 40d₄ of 1.59+/−0.03 millimeters. The lead connector pin 44 forms the electrical connection between the lead and the header 20.

The connector port or cavity 50 that is designed to fit with the pacemaker lead connector 40 is shown in FIG. 3. In one embodiment, the pacemaker lead connector port 50 also meets the requirement of ISO 5841-3 and is referred to as an IS-1 port. The lead connector port 50 has an orifice 25 that provides access for the lead connector 40 into the lead connector port 50. The lead connector port has a main body 52 with a diameter 50d₁ of 3.15+/−0.15 millimeters that, at the sealing ring zone 57 is 50d₂ 3.48+/−0.05 millimeters. Just past the sealing ring zone 57 the first shoulder 56 of the lead connector port 50 is formed. The lead connector port 50 tapers at the first shoulder 56 and at the second sealing zone 58, the diameter 50d₃ is 2.75+/−0.03 millimeters. Following the second sealing zone 58, a second shoulder 59 is formed in the lead connector port 50, proximate the lead connector pin port 54. The lead connector port 50 tapers again to form the lead connector pin port 54 that has a minimum diameter 50d₄ of 1.65 millimeters. Hence, the lead connector pin 44 of the pacemaker lead connector 40 fits through the lead connector pin port 54, allowing for the lead connector pin 44 to form a mechanical and electrical connection with the header 20.

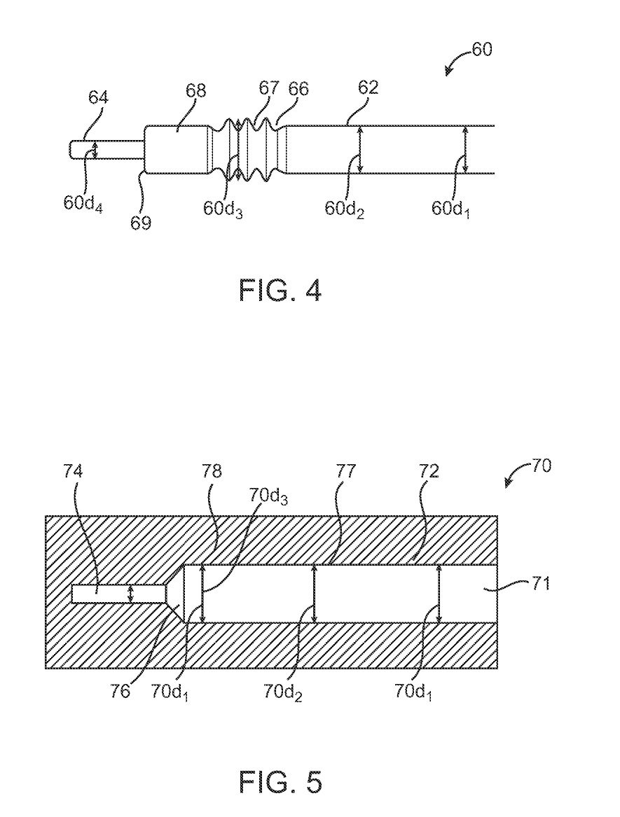

FIG. 4 is a cross-sectional view of the defibrillator lead connector 60 that meets the ISO 11318:2002 standard. The lead connector 60 for the cardiac lead 30 comprises a lead connector body 62 and a lead connector pin 64. The lead connector pin 64 is located at the proximal end of the lead connector 60 and, when locked in place in the port 70, forms a mechanical and physical connection between the lead connector 60 and the header 20. The lead connector pin 64 is made of conductive material.

The lead connector body 62 has a number of different diameters, as the lead connector body 62 tapers towards the lead connector pin 64. The diameter 60d₁ of the lead connector body 62 proximate the lead is 3.23+/−0.1 millimeters. The diameter of the main section of the lead connector body 62, 60d₂ is 3.23+0.1, −0.2 millimeters. This section of the lead connector body 62 extends up to the first shoulder 66, where at least one sealing ring 67 is located. At the first shoulder 66, the lead connector body 62 tapers slightly and then expands to accommodate the at least one sealing ring to a diameter 60d₃ of 3.36+/−0.01 millimeters. A second sealing area with at least one sealing ring 68 precedes the second shoulder 69 of the lead connector body 62. At the second shoulder 69, the lead connector body 62 tapers again such that the lead connector pin 64 is formed with a diameter 60d₄ of 1.25+/−0.03 millimeters. The lead connector pin 64 forms the electrical connection between the lead and the header 20.

The connector port or cavity 70 that is designed to fit with the defibrillator lead connector 60 is shown in FIG. 5. In one embodiment, the defibrillator lead connector port 70 also meets the requirement of ISO 11318:2002(E) and is referred to as a DF-1 port. The lead connector port 70 has an orifice 25 that provides access for the lead connector 60 into the lead connector port 70. The lead connector port 70 has a main body 72 with a minimum diameter 70d₁ of 3.43+/−0.15 millimeters that, at the sealing ring zone 77 is 70d₂ 3.48+/−0.05 millimeters. Just past the sealing ring zone 77 the first shoulder 76 of the lead connector port 70 is formed. The lead connector port 70 tapers at the first shoulder 76. However, just prior to the first shoulder 76, and at the second sealing zone 78, the diameter 70d₃ is 3.5+/−0.25 millimeters. Following the second sealing zone 78, the first shoulder 76 is formed in the lead connector port 70, proximate the lead connector pin port 74. The lead connector port 70 tapers to form the lead connector pin port 74 that has a diameter 70d₄ of 1.31 millimeters. Hence, the lead connector pin 64 of the defibrillator lead connector 60 fits through the lead connector pin port 74, allowing for the lead connector pin 64 to form a mechanical and electrical connection with the header 20.

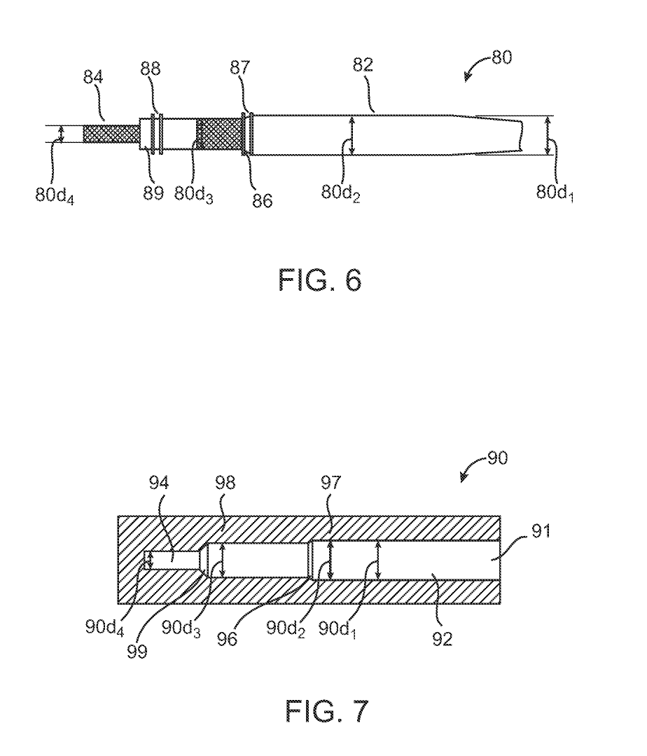

FIG. 6 is a cross-sectional view of a preferred embodiment of a non-cardiac lead connector 80 for a non-cardiac lead 32. The non-cardiac lead connector 80 comprises a lead connector body 82 and a lead connector pin 84. The lead connector pin 84 is located at the proximal end of the lead connector 80 and, when locked in place in the non-cardiac lead port 90, forms a mechanical and physical connection between the non-cardiac lead connector 80 and the header 20. The lead connector pin 84 is made of conductive material.

The lead connector body 82 has a number of different diameters, as the lead connector body 82 tapers towards the lead connector pin 84. The diameter 80d₁ of the lead connector body 82 proximate the lead is 3.1+/−0.3 millimeters. The diameter of the main section of the lead connector body 82, 80d₂ is 3.23+/−0.1 millimeters. This section of the lead connector body 82 extends up to the first shoulder 86, where at least one sealing ring 87 is located. At the first shoulder 86, the lead connector body 82 tapers to a diameter 80d₃ of 2.66+/−0.03 millimeters to 2.66+/−0.05 millimeters. A second sealing area with at least one sealing ring 88 precedes the second shoulder 89 of the lead connector body 82. At the second shoulder 89, the lead connector body 82 tapers again such that the lead connector pin 84 is formed with a diameter 80d₄ of 1.410+/−0.013 millimeters. The lead connector pin 84 forms the electrical connection between the lead and the header 20. As will be seen from a comparison of the lead connector body 82 of the non-cardiac lead 32 with the lead connector body 42 of the pacemaker IS-1 lead 30, all of the other dimensions up to the lead connector pin 84 are generally consistent with the dimensions of the IS-1 lead connector body 42.

The non-cardiac lead connector port or cavity 90 that is designed to fit with the non-cardiac lead connector 80 is shown in FIG. 7. The non-cardiac lead connector port 90 has an orifice 91 that provides access for the lead connector 80 into the lead connector port 90. The lead connector port 90 has a main body 92 with a diameter 90d₁ of 3.15+/−0.15 millimeters that, at the sealing ring zone 97 is 90d₂ 3.48+/−0.05 millimeters. Just past the sealing ring zone 97 the first shoulder 96 of the lead connector port 90 is formed. The lead connector port 90 tapers at the first shoulder 96 and at the second sealing zone 98, where the diameter 90d₃ is 2.75+/−0.03 millimeters. Following the second sealing zone 98, a second shoulder 99 is formed in the lead connector port 90, proximate the lead connector pin port 94. The lead connector port 90 tapers again to form the lead connector pin port 94 that has a diameter 90d₄ of 1.50+/−0.02 millimeters. Hence, the lead connector pin 84 of the non-cardiac lead connector 80 fits through the lead connector pin port 94, allowing for the lead connector pin 94 to form a mechanical and electrical connection with the header 20.

The non-cardiac lead connector pin 84 has been designed to have a diameter that is intermediate the defibrillator (DF-1) lead connector pin diameter and the pacemaker (IS-1) lead connector pin diameter. The ranges of diameters for lead connector pins and lead connector pin ports for the defibrillation lead (DF-1), the pacemaker lead (IS-1) and a preferred embodiment of a non-cardiac lead are provided in Table 1.

TABLE 1

| | | LEAD CONNECTOR |
|---|---|---|
| | LEAD CONNECTOR | PIN PORT |
| | PIN DIAMETER | DIAMETER |
| | (mm) | (mm) |
| DEFIBRILLATOR DF-1 | 1.25 ± 0.03 | 1.31 |
| PACEMAKER IS-1 | 1.59 ± 0.03 | 1.65 minimum |
| NON-CARDIAC (e.g., | 1.410 ± 0.013 | 1.50 ± 0.02 |
| BAROREFLEX | | |
| ACTIVATION | | |
| DEVICE) | | |

An advantage derived from the design of the non-cardiac lead connector 80 and corresponding port 90 is that an effective lockout connector arrangement is provided between the non-cardiac lead 32 and any standardized cardiac leads 30 for the implantable medical devices noted above. Due to the size of the diameter of the non-cardiac lead connector pin 84, the non-cardiac lead 32 cannot be mated with the defibrillator lead connector pin port 70. Since this connection is prevented, the possibility of high-energy defibrillation pulses inducing localized tissue damage, or worse trauma, is effectively eliminated. Another advantage derived from the configuration of the non-cardiac lead connector 80 and the corresponding port 90 is that the pacemaker lead connector (IS-1) pin 44 cannot be operably coupled with the non-cardiac lead connector pin port 94. Hence, the possibility of baroreflex activation therapies, for example, causing harm because they were delivered to cardiac tissue through a pacing lead (IS-1) also has been effectively eliminated as a result of the design in accordance with the present invention.

While the present invention has been described with respect to particular standards for the cardiac leads 30 and to one embodiment of a non-cardiac lead 32 for baroreflex activation proximate the carotid sinus, it is to be understood that variations in the present invention can be made without departing from the novel aspects of this invention as defined in the claims. For example, it is not necessary for an implantable pulse generator to have one or both of connector ports 50 (pacing) and 70 (defibrillation), such as in the case where the implantable pulse generator is solely designed for non-cardiac stimulation/sensing purposes. Alternatively, an implantable pulse generator which combined one or both of pacing and defibrillation therapies with a non-cardiac therapy, such as nerve stimulation, would have one or both of the connector ports (50) and 70 (defibrillation) in conjunction with the non-cardiac port 90 in accordance with the present invention.

What is claimed is:

1. A kit, comprising:
an implantable medical device having a header;
the header comprising at least one cardiac port and at least one non-cardiac port;
at least one cardiac electrode, each cardiac electrode being electrically connected to a cardiac connector;
at least one non-cardiac electrode, each non-cardiac electrode being electrically connected to a non-cardiac connector;
wherein the at least one non-cardiac port will not form a functional connection with the cardiac connector; and
wherein the at least one cardiac port will not form a functional connection with the non-cardiac connector.

2. The kit of claim 1, wherein:
wherein the at least one non-cardiac port will not receive the cardiac connector; and
wherein the at least one cardiac port will not receive the non-cardiac connector.

3. The kit of claim 1, wherein the at least one cardiac electrode is placed on or in the heart.

4. The kit of claim 1, wherein the at least one non-cardiac electrode is not placed on or in the heart.

5. The kit of claim 1, wherein the at least one non-cardiac connector comprises a lead connector pin having a diameter larger than an ISO standard DF-1 defibrillation lead connector pin.

6. The kit of claim 1, wherein the at least one non-cardiac connector comprises a lead connector pin having a diameter smaller than an ISO standard IS-1 pacemaker lead connector pin.

7. The kit of claim 1, wherein the at least one non-cardiac port comprises an orifice with a proximal-most portion having a diameter smaller than an ISO standard IS-1 pacemaker lead connector pin.

8. The kit of claim 1, wherein the at least one cardiac port comprises an orifice with a proximal-most portion having a diameter smaller than a diameter of a lead connector pin of the non-cardiac connector.

9. The kit of claim 1, wherein the at least one non-cardiac port comprises an orifice with a proximal-most portion having a diameter larger than an ISO standard DF-1 defibrillation lead connector pin.

10. The kit of claim 1, wherein a lead connector pin of the at least one non-cardiac connector has a diameter that is sufficiently large to prevent the non-cardiac connector from being received in an ISO standard DF-1 pin socket.

11. The kit of claim 1, wherein a lead connector pin of the non-cardiac connector has a diameter smaller than 1.56 millimeters.

12. The kit of claim 1, wherein a lead connector pin of the non-cardiac connector has a diameter larger than 1.31 millimeters.

13. The kit of claim 1, wherein the non-cardiac port has a distal diameter small enough to preclude insertion of at least some ISO standard DF-1 connector lead bodies.

14. The kit of claim 1, wherein the non-cardiac port has a proximal diameter small enough to preclude insertion of an ISO standard IS-1 connector pin.

15. The kit of claim 1, wherein the non-cardiac port has a distal diameter smaller than 3.21 millimeters.

16. The kit of claim 1, wherein the non-cardiac port has a proximal diameter smaller than 1.56 millimeters.

17. The kit of claim 1, wherein the at least one cardiac port comprises a pacing port dimensioned in accordance with ISO-5841-3.

18. The kit of claim 1, wherein the at least one cardiac port comprises a defibrillation port dimensioned in accordance with ISO-11318.

19. A system, comprising:
an implantable medical device having a header;
the header comprising a cardiac port and a non-cardiac port;
a cardiac connector received in a cardiac port of the header;
a cardiac electrode electrically connected to the cardiac connector;
a non-cardiac connector received in the non-cardiac port of the header;
a non-cardiac electrode electrically connected to a non-cardiac connector;
wherein the non-cardiac port will not form a functional connection with the cardiac connector; and
wherein the cardiac port will not form a functional connection with the non-cardiac connector.

20. The system of claim 19, wherein:
wherein the at least one non-cardiac port will not receive the cardiac connector; and
wherein the at least one cardiac port will not receive the non-cardiac connector.

21. The system of claim 19, wherein the at least one cardiac electrode is placed on or in the heart.

22. The system of claim 19, wherein the at least one non-cardiac electrode is not placed on or in the heart.

23. The system of claim 19, wherein the non-cardiac connector comprises a lead connector pin having a diameter larger than an ISO standard DF-1 defibrillation lead connector pin.

24. The system of claim 19, wherein the non-cardiac connector comprises a lead connector pin having a diameter smaller than an ISO standard IS-1 pacemaker lead connector pin.

25. The system of claim 19, wherein the non-cardiac port comprises an orifice with a proximal-most portion having a diameter smaller than the ISO standard IS-1 pacemaker lead connector pin.

26. The system of claim 19, wherein the cardiac port comprises an orifice with a proximal-most portion having a diameter smaller than a diameter of a lead connector pin of the non-cardiac connector.

27. The system of claim 19, wherein the non-cardiac port comprises an orifice with a proximal-most portion having a diameter larger than the ISO standard DF-1 defibrillation lead connector pin.

28. The system of claim 19, wherein a lead connector pin of the non-cardiac connector has a diameter that is sufficiently large to prevent the non-cardiac connector from being received in an ISO standard DF-1 pin socket.

29. The system of claim 19, wherein a lead connector pin of the non-cardiac connector has a diameter smaller than 1.56 millimeters.

30. The system of claim 19, wherein a lead connector pin of the non-cardiac connector has a diameter larger than 1.31 millimeters.

31. The system of claim 19, wherein the non-cardiac port has a distal diameter small enough to preclude insertion of at least some ISO standard DF-1 connector lead bodies.

32. The system of claim 19, wherein the non-cardiac port has a proximal diameter small enough to preclude insertion of an ISO standard IS-1 connector pin.

33. The system of claim 19, wherein the non-cardiac port has a distal diameter smaller than 3.21 millimeters.

34. The system of claim 19, wherein the non-cardiac port has a proximal diameter smaller than 1.56 millimeters.

35. The system of claim 19, wherein the cardiac port comprises a pacing port dimensioned in accordance with ISO-5841-3.

36. The system of claim 19, wherein the cardiac port comprises a defibrillation port dimensioned in accordance with ISO-11318.

37. A method of preventing patient harm or device damage in an implantable medical device including both cardiac and non-cardiac electrodes, the method comprising:
providing one or more cardiac electrodes, each cardiac electrode being electrically connected to a cardiac connector;
providing one or more non-cardiac electrodes, each non-cardiac electrode being electrically connected to a non-cardiac connector;
providing an implantable medical device including one or more cardiac ports and one or more non-cardiac ports;
wherein the one or more non-cardiac ports will not receive the cardiac connector; and
wherein the one or more cardiac ports will not receive the non-cardiac connector.
